(19) United States
(12) Patent Application Publication
(10) Pub. No.: US 2005/0234018 A1
(43) Pub. Date: Oct. 20, 2005

(54) Drug delivery to the back of the eye

(75) Inventors: Robert T. Lyons, Laguna Hills, CA (US); Chin-Ming Chang, Tustin, CA (US); Joan-En Chang-Lin, Tustin, CA (US); James Chang, Newport Beach, CA (US); Orest Olejnik, Coto De Caza, CA (US)

Correspondence Address: ALLERGAN, INC., LEGAL DEPARTMENT, 2525 DUPONT DRIVE, T2-7H, IRVINE, CA, 92612-1599, US

(73) Assignee: ALLERGAN, INC., Irvine, CA (US)

(21) Appl. No.: 10/826,843

(22) Filed: Apr. 15, 2004

(51) Int. Cl. A61K 31/724

(52) U.S. Cl. 514/58

(57) ABSTRACT

Disclosed herein are methods of delivering drugs or therapeutically active agents to the back of the eye via topical administration of compositions comprising cyclodextrin derivatives. Compositions related thereto are also disclosed herein.

### FIELD OF THE INVENTION

The present invention relates to pharmaceutical compositions. In particular, the present invention relates to compositions comprising prednisolone and prodrugs thereof.

### BACKGROUND OF THE INVENTION

1. Description of Related Art

Prednisolone is a potent corticosteroid which is effective in the treatment of a number of medical conditions. For certain indications, where passage of the drug through a lipid barrier is required, prodrugs with increased lipophilicity are often formulated to improve bioavailability. However, this complicates the formulation of aqueous liquid dosage forms. For example, prednisolone acetate, a commonly used lipophilic prednisolone prodrug, is not currently available in solution form, but is available as a suspension. Unfortunately, particularly in the case of ophthalmic formulations, using the compound in suspension form is believed to hamper the bioavailability of the prednisolone, thus attenuating the benefits associated with the use of a lipophilic prodrug. As such, the preparation of an aqueous composition of a completely dissolved lipophilic prednisolone prodrug would be a significant contribution to the art.

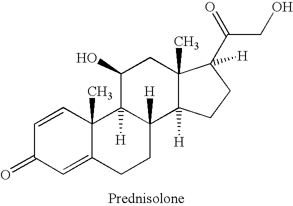

Cyclodextrins are cyclic oligosaccharides containing 6, 7, or 8 glucopyranose units, referred to as a-cyclodextrin (structure depicted below), β-cyclodextrin, or γ-cyclodextrin respectively, which are often used in pharmaceutical formulations. 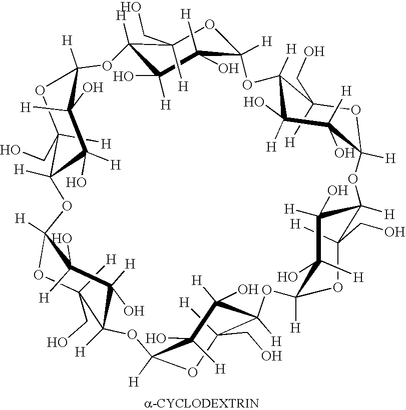
Cyclodextrins have a hydrophilic exterior, which makes them water-soluble, and a hydrophobic interior which forms a cavity. In an aqueous environment, hydrophobic portions of molecules often enter the hydrophobic cavity of cyclodextrin to form inclusion compounds. Although inclusion compounds are often formed between cyclodextrins and hydrophobic molecules, cyclodextrins are also capable of other types of nonbonding interactions with molecules that are not inside the hydrophobic cavity. Cyclodextrins have three free hydroxyl groups for each glucopyranose unit, or 18 hydroxyl groups on α-cyclodextrin, 21 hydroxyl groups on β-cyclodextrin, and 24 hydroxyl groups on γ-cyclodextrin. One or more of these hydroxyl groups can be reacted with any of a number of reagents to form a large variety of cyclodextrin derivatives. Some of the more common derivatives of cyclodextrin are hydroxypropyl ethers, sulfonates, and sulfoalkylethers.

In pharmaceutical formulations, cyclodextrins and cyclodextrin derivatives are often used to improve the solubility of a drug. While inclusion compounds are involved in many cases of enhanced solubility, other interactions between cyclodextrins and insoluble compounds can also improve solubility. As mentioned, the use of cyclodextrins in pharmaceutical compositions is well known in the art. For example, U.S. Pat. No. 6,407,079 teaches the use of β-cyclodextrin derivatives to form inclusion compounds that improve the solubility of the drug

U.S. Pat. No. 5,472,954 and EP 579435 teach “the use of certain polymers in the preparation of cyclodextrin-drug complexes as a means for increasing the solubilizing and stabilizing effects of cyclodextrin derivatives on drugs,” specifying that “from about 0.001% to about 5%” of said polymers are useful in this respect. Furthermore, the patents require that the polymer and cyclodextrin be dissolved together before the addition of the drug, and that the polymer, cyclodextrin, and the drug be heated together. The '954 patent also discloses the use of hydroxypropylmethylcellulose and hydroxypropyl cyclodextrins to solubilize hydrocortisone.

The use of cyclodextrin and cyclodextrin derivatives in ophthalmic formulations is also known. For example, EP 0435682 A2 teaches the use of cyclodextrins in ophthalmic compositions with prostaglandins to treat ocular hypertension.

In the selection of cyclodextrin and cyclodextrin derivatives for pharmaceutical and other applications, β-cyclodextrin and its derivatives appear to be favored over the other cyclodextrins. For example, EP 0794783 B1 states “β-cyclodextrin has been of special interest because of its cavity size”.

In citing the foregoing references, and other references cited herein, applications make no admission as to whether any of said references constitutes prior art. Rather, the determination of what constitutes prior art is a legal decision made on the basis of the dates said references were made available to the public, the authors or inventors of said references, and the effective filing date of the disclosure made herein.

### SUMMARY OF THE INVENTION

### BRIEF DESCRIPTION OF THE INVENTION

A method comprising topically administering a composition comprising a cyclodextrin and a therapeutically active agent, or a pharmaceutically acceptable salt or a prodrug thereof, to the eye of a mammal in need thereof, wherein said method is effective in improving delivery of said therapeutically active agent to the back of the eye is disclosed herein.

Another embodiment comprises a pharmaceutical product comprising a solution comprising a therapeutically active agent, or a salt or a prodrug thereof, and a cyclodextrin, wherein said solution has an ophthalmically acceptable pH,
a container suitable for dispensing drops of said solution to the eye of a mammal in need of treatment by said prodrug, and
a package which indicates that said product is useful for treatment of a disease or condition affecting the back of the eye.

Use of a combination of a therapeutically active agent and a cyclodextrin in the manufacture of a medicament for the treatment of a condition or disease affecting the back of the eye is also disclosed herein.

A composition comprising a therapeutically active agent and a cyclodextrin, wherein said therapeutically active agent is intended for treatment or prevention of a disease or condition affecting the back of the eye, and wherein said composition is suitable for topical ophthalmic administration is also disclosed herein.

### BRIEF DESCRIPTION OF THE DRAWING FIGURES

FIG. 1 is a plot showing the concentration of prednisolone in the aqueous humor of rabbit eyes after topical administration of the compositions of formula 1a-1e to the eyes of the animals.

FIG. 2 is a plot showing the concentrations of prednisolone and prednisolone acetate in the aqueous humor of rabbit eyes after topical administration of the compositions of formula 2a-2g to the eyes of the animals.

FIG. 3 is a plot showing the concentrations of prednisolone in the vitreous humor of rabbit eyes after topical administration of the compositions of formula 2a-2g to the eyes of the animals.

FIG. 4 is a plot comparing the concentration of prednisolone in the aqueous humor (AH) to that of the vitreous humor, multiplied for ease of comparison [VH (×65)], after topical administration of the compositions of formula 2a-2g to the eyes of the animals.

FIG. 5 is a plot of the tonicity of a solution of β-cyclodextrin (β-CD), hydroxypropyl-γ-cyclodextrin (HPCD), sulfobutylether-β-cyclodextrin (CaSBECD) calcium salt, and sulfobutylether-β-cyclodextrin (NaSBECD) sodium salt at various concentrations in aqueous solution.

FIG. 6 is a plot of the solubility of prednisolone acetate in various hydroxypropyl-γ-cyclodextrin (HPγCD) solutions with and without hydrophilic polymers.

FIG. 7 is a plot of the solubility of prednisolone acetate in an aqueous 25% hydroxypropyl-γ-cyclodextrin solution in the presence of varying amounts of hydroxypropylmethylcelluse (HPMC).

### DETAILED DESCRIPTION OF THE INVENTION

We have surprisingly discovered that cyclodextrins are effective in improving delivery of therapeutically active agents to the back of the eye. This is accomplished by topically administering a composition which comprises a therapeutically active agent, or a pharmaceutically acceptable salt or a prodrug thereof, and a cyclodextrin.

Compositions for use in the methods and products disclosed herein comprise a therapeutically active agent, or a pharmaceutically acceptable salt or a prodrug thereof, and a cyclodextrin.

These composition and methods are practiced upon a mammal “in need thereof”. In other words, these compositions and methods are practiced upon a mammal who is suffering from, or at risk of suffering from, a disease or condition affecting the back of the eye.

A therapeutically active agent is a compound which is useful in treating or preventing a condition or a disease which afflicts a mammal. In other words said condition or disease is associated with undesirable effects in said mammal. While not intending to be limiting, examples of therapeutically active agents include retinoids, prostaglandins, tyrosine kinase inhibitors, adrenoreceptor agonists or antagonists, dopaminergic agonists, cholinergic agonists, carbonic anhydrase inhibitors, guanylate cyclase activators, cannabinoids, endothelin, adenosine agonists, and neuroprotectants; analgesics/antipyretics such as aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, and meprobamate; antibiotics such as neomycin, streptomycin, chloramphenicol, cephalosporin, ampicillin, penicillin, and tetracycline; antidepressants such as nefopam, oxypertine, doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, desipramine hydrochloride, trimipramine maleate, and protriptyline hydrochloride; antidiabetics such as biguanides, hormones, and sulfonylurea derivatives; antihypertensive agents such as propanolol, propafenone, oxyprenolol, Nifedipine, reserpine, trimethaphan camsylate, phenoxybenzamine hydrochloride, pargyline hydrochloride, deserpidine, diazoxide, guanethidine monosulfate, minoxidil, rescinnamine, sodium nitroprusside, rauwolfia serpentina, alseroxylon, phentolamine mesylate, and reserpine; anti-inflammatories such as indomethacin, naproxen, ibuprofen, ramifenazone, piroxicam, cortisone, dexamethasone, fluazacort, hydrocortisone, prednisolone, and prednisone; antineoplastics such as adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, and piposulfan; antianxiety agents such as lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, and dantrolene; immunosuppressive agents such as cyclosporine, azathioprine, mizoribine, and tacrolimus; antimigraine agents such as ergotamine tartrate, propanolol hydrochloride, isometheptene mucate, and dichloralphenazone; antianginal agents such as beta-adrenergic blockers, nifedipine, diltiazem hydrochloride nitrates, nitroglycerin, isosorbide dinitrate, pentaerythritol tetranitrate, erythrityl and tetranitrate; antipsychotic agents such as haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, and prochlorperazine; antimanic agents such as lithium carbonate; antiarrhythmics such as bretylium tosylate, esmolol hydrochloride, verapamil hydrochloride, amiodarone, encainide hydrochloride, digoxin, digitoxin, mexiletine hydrochloride, disopyramide phosphate, procainamide hydrochloride, quinidine sulfate, quinidine gluconate, quinidine polygalacturonate, flecainide acetate, tocainide hydrochloride, and lidocaine hydrochloride; antiarthritic agents such as phenylbutazone, sulindac, penicillamine, salsalate, piroxicam, azathioprine, indomethacin, meclofenamate sodium, gold sodium thiomalate, ketoprofen, auranofin, aurothioglucose, and tolmetin sodium; antigout agents such as colchicine and allopurinol; anticoagulants such as heparin, heparin sodium, and warfarin sodium; thrombolytic agents such as urokinase, streptokinase, and altoplase; antifibrinolytic agents such as aminocaproic acid; hemorheologic agents such as pentoxifylline; antiplatelet agents such as aspirin, empirin, and ascriptin; anticonvulsants such as valproic acid, divalproate sodium, phenytoin, phenytoin sodium, clonazepam, primidone, phenobarbitol, phenobarbitol sodium, carbamazepine, amobarbital sodium, methsuximide, metharbital, mephobarbital, mephenytoin, phensuximide, paramethadione, ethotoin, phenacemide, secobarbitol sodium, clorazepate dipotassium, and trimethadione; antiparkinson agents such as ethosuximide; antihistamines/antipruritics such as loradatine, hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, and trimprazine tartrate; agents useful for calcium regulation such as calcitonin and parathyroid hormone; antibacterial agents such as amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate; antiviral agents such as interferon gamma, zidovudine, amantadine hydrochloride, ribavirin, and acyclovir; antimicrobials such as cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G potassium, penicillin G procaine, methicillin sodium, nafcillin sodium, erythromycin ethylsuccinate, erythromycin, erythromycin estolate, erythromycin lactobionate, erythromycin siearate, erythromycin ethylsuccinate, tetracycline hydrochloride, doxycycline hyclate, and minocycline hydrochloride; anti-infectives such as GM-CSF; bronchodialators such as epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate), anticholinergic agents, aminophylline, dyphylline, metaproterenol sulfate, aminophylline, mast cell stabilizers, flurisolidebeclomethasone dipropionate, beclomethasone dipropionate monohydrate, salbutamol, beclomethasone dipropionate, ipratropium bromide, budesonide, ketotifen, salmeterol, xinafoate, terbutaline sulfate, triamcinolone, theophylline, nedocromil sodium, metaproterenol sulfate, albuterol, and flunisolide; hormones such as danazol, testosterone cypionate, fluoxymesterone, ethyltostosterone, testosterone enanihate, methyltestosterone, fluoxymesterone, testosterone cypionate, estradiol, estropipate, conjugated estrogens, methoxyprogesterone acetate, norethindrone acetate, triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate, and thyroid hormones; hypoglycemic agents such as human insulin, purified beef insulin, purified pork insulin, glyburide, chlorpropamide, glipizide, tolbutamide, and tolazamide; hypolipidemic agents such as clofibrate, dextrothyroxine sodium, probucol, lovastatin, and niacin; and agents useful for erythropoiesis stimulation such as erythropoietin. In one embodiment, the therapeutically active agent is water-insoluble, meaning that its solubility in water at room temperature is less than 0.1%. In another embodiment, the therapeutically active agent is water-soluble, meaning that it has a solubility in water at room temperature of greater than or equal to 0.1%.

The following types of therapeutically active agents are of particular interest in the treatment of diseases affecting the back of the eye: retinoids, prostaglandins, alpha-2-adrenergic agonists, beta adrenoreceptor antagonists, dopaminergic agonists, cholenergic agonists, tyrosine kinase inhibitors, antiinflammatories, corticosteroids, NMDA antagonists, anti-cancer drugs and antihistamines.

Another therapeutically active agent that is useful in treating diseases affecting the back of the eye is memantine.

The term “cyclodextrin” as disclosed herein should be interpreted broadly to include the natural cyclodextrins and their derivatives, including the alkylated and hydroxyalkylated derivatives and the branched cyclodextrins. Cyclodextrins and their derivatives which have been previously described as useful for complexation with drugs are of particular interest herein. In addition to α-, β- and γ-cyclodextrin, the ether and mixed ether derivatives and those derivatives bearing sugar residues are of special interest. Especially useful herein are the hydroxyethyl, hydroxypropyl (including 2- and 3-hydroxypropyl) and dihydroxypropyl ethers, their corresponding mixed ethers and further mixed ethers with methyl or ethyl groups, such as methyl-hydroxyethyl, ethyl-hydroxyethyl and ethyl-hydroxypropyl ethers of α-, β- and γ-cyclodextrin. Hydroxypropyl-β-cyclodextrin and its preparation by propylene oxide addition to β-cyclodextrin, and hydroxyethyl-β-cyclodextrin and its preparation by ethylene oxide addition to β-cyclodextrin, were described in a patent of Gramera et al. (U.S. Pat. No. 3,459,731, issued August 1969) over 20 years ago. Other useful cyclodextrin derivatives are maltosyl, glucosyl and maltotriosyl derivatives of β-and γ-cyclodextrin, which may contain one or more sugar residues, e.g. glucosyl or diglucosyl, maltosyl or dimaltosyl, as well as various mixtures thereof, e.g. a mixture of maltosyl and dimaltosyl derivatives. Other useful cyclodextrin derivatives comprise anionic functional groups such as sulfobutylether derivatives, sulfonates, phosphates, and the like. Specific examples of cyclodextrin derivatives for use herein include hydroxypropyl-β-cyclodextrin, hydroxypropyl-γ-cyclodextrin, sulfobutylether-β-cyclodextrin, and sulfobutylether-γ-cyclodextrin, as well as hydroxyethyl-β-cyclodextrin, hydroxyethyl-γ-cyclodextrin, dihydroxypropyl-β-cyclodextrin, glucosyl-β-cyclodextrin, diglucosyl-β-cyclodextrin, maltosyl-β-cyclodextrin, maltosyl-γ-cyclodextrin, maltotriosyl-β-cyclodextrin, maltotriosyl-γ-cyclodextrin and dimaltosyl-β-cyclodextrin, and mixtures thereof such as maltosyl-β-cyclodextrin/dimaltosyl-βcyclodextrin. Procedures for preparing such cyclodextrin derivatives are well-known, for example, from Bodor U.S. Pat. No. 5,024,998, dated Jun. 18, 1991, expressly incorporated herein by reference, and references cited therein.

In certain circumstances, it may be desirable to use a cyclodextrin derivative. The term “cyclodextrin derivative” has the broadest meaning generally understood in the art, and refers to a compound or a mixture of compounds wherein one or more of the free hydroxyl groups of α-, β-, or γ-cyclodextrin is replaced with any other group. A “water-soluble” cyclodextrin derivative is soluble at a concentration of at least 300 mg/mL in water. The cyclodextrin derivative used in the compositions disclosed herein may vary. Derivatives of α-cyclodextrin, β-cyclodextrin, and γ-cyclodextrin may be used. In certain compositions, a β-cyclodextrin derivative such as calcium sulfobutylether-β-cyclodextrin, sodium sulfobutylether-β-cyclodextrin, and hydroxypropyl-β-cyclodextrin, may be used. Alternatively, a γ-cyclodextrin derivative such as calcium sulfobutylether-γ-cyclodextrin, sodium sulfobutylether-γ-cyclodextrin, and hydroxypropyl-γ-cyclodextrin may be used. Specifically contemplated herein are the hydroxypropyl derivatives of cyclodextrins, such as hydroxypropyl-β-cyclodextrin or hydroxypropyl-γ-cyclodextrin.

The concentration of the cyclodextrin used in the compositions and methods disclosed herein can vary according to the physico-chemical properties, pharmacokinetic properties, side effect or adverse events, formulation considerations, or other factors associated with the therapeutically active agent, or a salt or prodrug thereof. The properties of other excipients in a composition may also be important. Thus, the concentration or amount of cyclodextrin used in accordance with the compositions and methods disclosed herein can vary. In certain compositions, the concentration of the cyclodextrin is from 10% to 25%. In other embodiments, the concentration of the cyclodextrin is greater than 10%. In certain liquid compositions the concentration of the cyclodextrin is above 10% and less than 40%. In other compositions, the concentration of the cyclodextrin is from about 1% to about 30%. In other compositions, the concentration of the cyclodextrin is from about 10% to about 30%. In other compositions, the concentration of the cyclodextrin is from 20% to 25%. In other compositions, the concentration of the cyclodextrin is about 10%. In other compositions, the concentration of the cyclodextrin is about 15%. In other embodiments, the concentration of the cyclodextrin is about 25%. In other compositions, the concentration of the cyclodextrin is about 30%.

One composition comprises from 5% to 35% of hydroxypropyl-β-cyclodextrin or hydroxypropyl-γ-cyclodextrin.

A “pharmaceutically acceptable salt” is any salt that retains the activity of the parent compound and does not impart any additional deleterious or untoward effects on the subject to which it is administered and in the context in which it is administered compared to the parent compound.

Pharmaceutically acceptable salts of acidic functional groups may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted.

A “water-insoluble” prodrug is a prodrug which is not soluble at a therapeutically effective concentration in an aqueous liquid composition. A “nonionic” prednisolone prodrug is a prodrug having no ionic groups such as phosphate, sulfate or carboxylate. On example of a prodrug which is useful for the compositions disclosed herein is prednisolone acetate, which has the structure shown below.

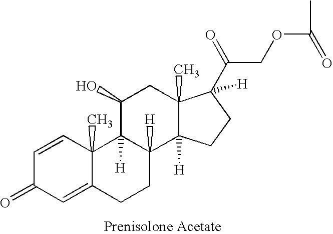

The determination of a therapeutically effective concentration of prednisolone or a prodrug thereof is well within the ability of a person having ordinary skill in the art. The meaning of “an effective concentration” should be interpreted broadly, and will vary widely depending on circumstances such as the condition being treated, the mammal to which the compound is being administered, the method of administration, formulation considerations, manufacturing considerations, preferences of those administering and being administered the compound, and convenience. One composition comprises about 0.5% prednisolone acetate. Another composition comprises greater than 0.5% prednisolone acetate. Another composition comprises about 0.4% prednisolone acetate. Another composition comprises from 0.1% to 1.5% prednisolone acetate. Another composition comprises from 0.2% to 0.7% prednisolone acetate. Another composition comprises from 0.6% to 1.6% prednisolone acetate. Another composition comprises about 0.6% prednisolone acetate. Another composition comprises about 1% prednisolone acetate. Another composition comprises about 1.2% prednisolone acetate.

In relation to the delivery of a therapeutically active agent to the back of the eye, the term “back of the eye” refers to any structure, or combination of structures of the eye which include the vitreous humor and anything posterior thereto including the uveal tract, retina, macula, fovia, choroid, optic nerve, retinal pigmented epithelium, etc. Any composition disclosed herein relevant to any of the other embodiments may be used in this method. In one embodiment, a solution comprising prednisolone acetate and hydroxpropyl-β-cyclodextrin is administered. In another embodiment, a solution comprising prednisolone acetate and hydroxypropyl-γ-cyclodextrin is administered.

Certain compositions comprise a water-soluble polymer. While not intending to limit the scope of the invention in any way, cellulose derivatives such as carboxymethylcellulose and hydroxypropylmethylcellulose are useful water-soluble polymers for certain of the compositions disclosed herein. One composition comprises less than 1% hydroxypropylmethylcellulose. Another composition comprises hydroxypropylmethylcellulose having a concentration less than 1%. Another composition comprises from 0% to 1% hydroxypropylmethylcellulose. Other compositions comprise from 0.05% to 0.4% hydroxypropylmethylcellulose. Another embodiment comprises about from 0.12% to 0.3% hydroxypropylmethylcellulose. Another embodiment comprises about from 0.1% to 0.25% hydroxypropylmethylcellulose. Another composition comprises from 0% to 0.15% hydroxypropylmethylcellulose.

While not intending to limit the scope of the invention in any way, topical ophthalmic formulations often comprises an effective amount of buffer necessary to maintain the pH at the desired range, one or more tonicity agents, a preservative, and a chelating agent.

Buffers are well known by those skilled in the art and some examples of useful buffers are acetate, borate, carbonate, citrate, and phosphate buffers. While not intending to limit the scope of the invention in any way, certain compositions disclosed herein have a pH of from 4 to 8. Other compositions have a pH of 4.5 to 5.5.

Tonicity agents are used to adjust the composition of the formulation to the desired isotonic range. Tonicity agents are well known in the art and some examples include glycerin, mannitol, sorbitol, sodium chloride, and other electrolytes.

Preservatives are used to prevent bacterial contamination in multiple-use ophthalmic preparations. Preservatives are well known in the art, and, while not intending to be limiting, examples include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof are examples of useful preservatives.

A chelating agent is often used in ophthalmic compositions to enhance preservative effectiveness. The term “chelating agent” has the meaning generally understood in the art, and while not intending to be limiting, suitable chelating agents include edetate salts like edetate disodium, edetate calcium disodium, edetate sodium, edetate trisodium, and edetate dipotassium.

Certain compositions disclosed herein comprise from 0.6% to 1.6% prednisolone acetate, from 10% to 25% hydroxypropyl-γ-cyclodextrin, from 0% to 0.15% hydroxypropylmethylcellulose, a buffer, and a chelating agent, wherein said composition is isotonically adjusted for ophthalmic administration, and said composition has a pH of from 4.5 to 5.5.

Another composition comprises about 0.4% prednisolone acetate, about 10% hydroxypropyl-β-cyclodextrin, and about 0.5% hydroxypropylmethylcellulose.

Another composition comprises from 0.1% to 1.5% prednisolone acetate, from 5% to 35% hydroxypropyl-β-cyclodextrin or hydroxypropyl-γ-cyclodextrin, and
from 0% to 1% hydroxypropylmethylcellulose.

In certain embodiments, the compositions disclosed herein are dispensed as drops from a container suitable for such a purpose. Such a container is any container that can be used to dispense individual drops of the composition, wherein the drops are of a size which is amenable for ophthalmic use.

In certain embodiments the therapeutically active agent is not administered to treat a condition affecting the front of the eye. In other words, the drug is not delivered to the back of the eye as a result of the topical administration of a drug to treat a condition affecting the front of the eye. Examples of such front of the eye conditions elevated intraocular pressure, allergic conjunctivitis, and dry eye. In one embodiment, the therapeutically active agent or a salt or prodrug thereof, is used for neuroprotection in a glaucoma patient, but is not used to reduce intraocular pressure.

In one embodiment, the mammal being treated is a human.

Some examples of the diseases or conditions affecting the back of the eye include, without limitation, the following:

MACULOPATHIES/RETINAL DEGENERATION: Non-Exudative Age Related Macular Degeneration (ARMD), Exudative Age Related Macular Degeneration (ARMD), Choroidal Neovascularization, Diabetic Retinopathy, Acute Macular Neuroretinopathy, Central Serous Chorioretinopathy, Cystoid Macular Edema, Diabetic Macular Edema.

UVEITIS/RETINITIS/CHOROIDITIS: Acute Multifocal Placoid Pigment Epitheliopathy, Behcet's Disease, Birdshot Retinochoroidopathy, Infectious (Syphilis, Lyme, Tuberculosis, Toxoplasmosis), Intermediate Uveitis (Pars Planitis), Multifocal Choroiditis, Multiple Evanescent White Dot Syndrome (MEWDS), Ocular Sarcoidosis, Posterior Scleritis, Serpignous Choroiditis, Subretinal Fibrosis and Uveitis Syndrome, Vogt-Koyanagi-Harada Syndrome.

VASCULAR DISEASES/EXUDATIVE DISEASES: Retinal Arterial Occlusive Disease, Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease (CAD), Frosted Branch Angitis, Sickle Cell Retinopathy and other Hemoglobinopathies, Angioid Streaks, Familial Exudative Vitreoretinopathy, Eales Disease.

TRAUMATIC/SURGICAL: Sympathetic Ophthalmia, Uveitic Retinal Disease, Retinal Detachment, Trauma, Laser, PDT, Photocoagulation, Hypoperfusion During Surgery, Radiation Retinopathy, Bone Marrow Transplant Retinopathy.

PROLIFERATIVE DISORDERS: Proliferative Vitreal Retinopathy and Epiretinal Membranes, Proliferative Diabetic Retinopathy.

INFECTIOUS DISORDERS: Ocular Histoplasmosis, Ocular Toxocariasis, Presumed Ocular Histoplasmosis Syndrome (POHS), Endophthalmitis, Toxoplasmosis, Retinal Diseases Associated with HIV Infection, Choroidal Disease Associated with HIV Infection, Uveitic Disease Associated with HIV Infection, Viral Retinitis, Acute Retinal Necrosis, Progressive Outer Retinal Necrosis, Fungal Retinal Diseases, Ocular Syphilis, Ocular Tuberculosis, Diffuse Unilateral Subacute Neuroretinitis, Myiasis.

GENETIC DISORDERS: Retinitis Pigmentosa, Systemic Disorders with Accosiated Retinal Dystrophies, Congenital Stationary Night Blindness, Cone Dystrophies, Stargardt's Disease and Fundus Flavimaculatus, Best's Disease, Pattern Dystrophy of the Retinal Pigmented Epithelium, X-Linked Retinoschisis, Sorsby's Fundus Dystrophy, Benign Concentric Maculopathy, Bietti's Crystalline Dystrophy, pseudoxanthoma elasticum.

RETINAL TEARS/HOLES: Retinal Detachment, Macular Hole, Giant Retinal Tear.

TUMORS: Retinal Disease Associated with Tumors, Congenital Hypertrophy of the RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, Intraocular Lymphoid Tumors.

MISCELLANEOUS: Punctate Inner Choroidopathy, Acute Posterior Multifocal Placoid Pigment Epitheliopathy, Myopic Retinal Degeneration, Acute Retinal Pigment Epithelitis and the like.

The best modes of making and using the present invention are described in the following examples. These examples are given only to provide direction and guidance in how to make and use the invention, and are not intended to limit the scope of the invention or be relevant thereto, in any way.

### EXAMPLE 1

Compositions comprising β-cyclodextrin derivatives disclosed in Table 1 were prepared by the following procedure. Part I was made by combining 3.15 g each of sodium acetate and acetic acid with 8993.7 g purified water in a 10 L bottle, stirring until dissolved, and then adjusting to pH 4.5 with acetic acid as needed. Part II was made by slowly adding 25.00 g HPMC to 1225.0 g Part I acetate buffer (10 mM) at 65° C. with propeller mixing. The heat was removed and mixing continued while the solution cooled to room temperature. The solution was refrigerated overnight to complete the hydration. Part III was made by weighing 1.00 g disodium EDTA into a 10 L media bottle. Part II (1250 g) was weighed into the 10 L media bottle containing Part III. Part I (acetate buffer, 6881.01 g) and the preservative (polyhexamethylenebiguanidine [PHMB], 1-4 mg) were weighed into the media bottle already containing Parts II and III and then mixed without heating until dissolved. Hydroxypropyl-β-cyclodextrin (2587.99 g) was added to a 20 liter stainless steel water-jacketed tank equipped with scraping and mixing devices (VME-20), and then the combined solution (Parts I, II, and III) containing acetate buffer, HPMC, and EDTA were added to the VME-20. The scraper was started at 50% speed to mix the ingredients until they were completely wetted, adjusting the speed as needed. A static vacuum was applied and the scraper speed was increased to 100%, and mixing was continued until all material was dissolved. The vacuum was then released, and the scraper stopped. Prednisolone acetate (130.00 g) was then added, and the mixture was mixed until dispersed with scraper at 100% speed and dissolver at 20% speed. Speeds were adjusted as needed to minimize airborne powder. A static vacuum was applied after the prednisolone acetate was wetted, and mixing was continued while heating the mixture to 120° C., the mixture was stirred at 120° C. for 20 minutes, cooled to 30° C. with mixing, and then mixed for 30 additional minutes after the mixture had reached 30° C.

TABLE 1

| Excipient | Formula 1a | Formula 1b | Formula 1c | Formula 1d | Formula 1e |
|---|---|---|---|---|---|
| Prednisolone acetate | 1.4% | 0.4% | 1.1% | 0.2% | 1.0% |
| Hydroxypropyl-β-cyclodextrin | 30% | 10% | 30 | — | 0 |
| Sulfobutylether-β-cyclodextrin | — | — | — | 10% | 0 |
| Hydroxypropylmethylcellulose | 0.5% | 0.5% | 0% | 0.5% | 0.12 |
| Acetate Buffer (pH 6) | 0.08% | 0.08% | 0.08% | 0.08% | 0 |
| Edetate disodium (EDTA) | 0.01% | 0.01% | 0.01% | 0.01% | 0.0127% |

% is % w/v

The bioavailability of prednisolone acetate in the formulations described above was assessed by topical ophthalmic administration of said formulations to rabbits. A single 35 μL dose was administered topically to the lower cul-de-sac of both eyes in female New Zealand white rabbits using two rabbits per sampling time for each of five treatment groups. Aqueous humor samples (100 μL) were collected from four eyes at 0.5, 1, 2, and 4 hours post-dosing. Prednisolone acetate, prednisolone and prednisone were extracted (300 μL methanol:acetonitrile, 50:50 v/v) from aqueous humor samples, and extracts were analyzed by a liquid chromatography tandem mass spectrometry (LC-MS/MS) method with a quantization range of 5-200 ng/mL.

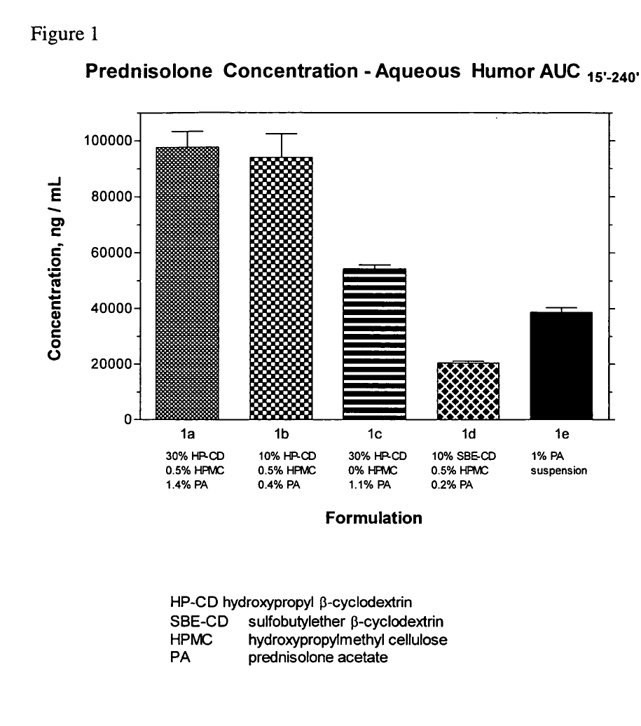

The total area under the curve (AUC) for each formulation is depicted in FIG. 1. These results surprisingly show that the β-cyclodextrin derivatives enhance the bioavailability of the drug in the aqueous humor. In almost every case, the concentration of the drug in the aqueous humor is higher for the formulations containing a β-cyclodextrin derivative compared to the control suspension, which contains no cyclodextrin or derivative thereof. The lone exception occurs in the case of the sulfobutylether-β-cyclodextrin. In that particular case, however, the active concentration in the formulation is only 20% that of the control (Formula 1e), whereas the concentration in the aqueous humor is about half that of the control, so there is approximately a 2.5-fold improvement in the bioavailability for the sulfobutylether-β-CD containing formulation as well.

While not intending to be limiting, these results also show that the water-soluble polymer (Formula 1c) is not required to improve the bioavailability of prednisolone acetate over the control. It also appears that in the case of β-cyclodextrin derivatives, the hydroxypropyl derivative is superior to the sulfobutylether derivative. While not intending to limit the scope of the invention, or to be bound in any way by theory, these results also show that over a two-fold enhancement of the bioavailability of the drug can be achieved with the compositions disclosed herein (Formulas 1a and 1b). Also, while not intending to limit the scope of the invention, for the combination of prednisolone acetate, hydroxypropyl-β-cyclodextrin, and hydroxypropylmethylcellulose, increasing the concentration of prednisolone acetate above 0.4% and the concentration of hydroxypropyl-β-cyclodextrin above 10% provides only minimal additional benefit. In conclusion, while not intending to be limited by theory, these results clearly show that the compositions provided herein represent a significant improvement over the current art in the topical ophthalmic delivery of prednisolone acetate to the aqueous humor.

### EXAMPLE 2

Compositions 2a-2c comprising γ-cyclodextrin derivatives described in Table 2 were prepared by the procedure of Example 4. Composition 2f, which contains HPβCD for comparison purposes, was also prepared by the procedure of Example 4. Compositons 2d and 2e were prepared by the procedure of Example 6. Composition 2g is a commercial formulation (Pred Forte® suspension, Allergan, Inc., Irvine, Calif.). In addition to the ingredients listed, compositions 2a-2f contained 0.05% EDTA, 2 ppm PHMB, had a pH of 4.8 and used NaCl as a tonicity agent if needed. Composition 2g, used as a control, contained 0.0127% EDTA, 60 ppm BAK, had a pH of 5.3, and used NaCl as a tonicity agent.

TABLE 2

| | Prednisolone | Hydroxypropyl-γ- | |
|---|---|---|---|
| | Acetate | cyclodextrin | Hydroxypropymethylcellulose |
| Formula | (% w/v) | (HPγCD) | (HPMC) |
| 2a | 1.1 | 25 | 0.12 |
| 2b | 0.5 | 15 | 0.12 |
| 2c | 0.6 | 25 | 0 |
| 2d | 1.0 | 25 | 0.12 |
| 2e | 1.0 | 25 | 0 |
| 2f | 1.2 | (30% | 0.5 |
| | | hydroxypropyl-β- | |
| | | cyclodextrin) | |
| 2g | 1.0 | —* | 0.12 |

*Commercial suspension

The relative ocular absorption of prednisolone acetate and its metabolites, 20 prednisolone and prednisone, from formulas 2a-2f were compared with that of formula 2g following a single 35 μL ophthalmic administration in New Zealand White rabbits (Table 2). Twenty-one female rabbits were given a single drop into both eyes and aqueous humor and vitreous humor samples were collected from animals (n=3 animals per formulation) at 60 minutes postdose. Prednisolone acetate, prednisolone and prednisone extracted from aqueous humor and vitreous humor samples were analyzed by a liquid chromatography tandem mass spectrometry (LC-MS/MS) method with a quantitation range of 5-200 ng/mL.

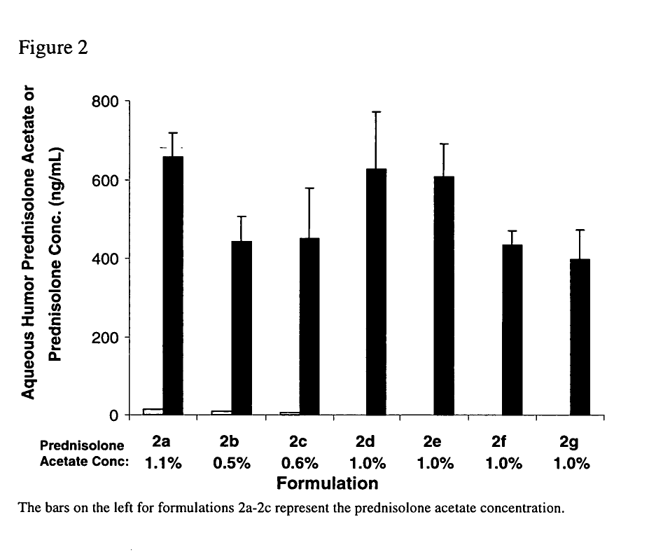

The aqueous humor concentration of prednisolone and prednisolone acetate for each of the compositions of Table 2 is depicted in FIG. 2. While not intending to be bound in any way by theory, the compositions containing cyclodextrin clearly delivered the drug to the aqueous humor better than the commercial formulation, which contains no cyclodextrin.

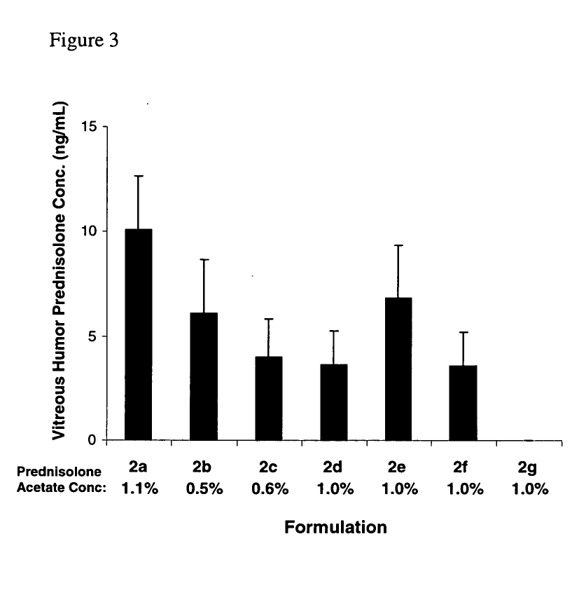

While not intending to limit the scope of the invention in any way, or to be bound in any way by theory, we have surprisingly discovered that cyclodextrin derivatives significantly enhance passage of prednisolone from the aqueous humor to the vitreous humor. FIG. 3 summarizes the vitreous humor concentration of prednisolone for the compositions of Table 2. The cyclodextrin-derivative containing formulations (2a-2f) clearly delivered significantly more drug to the vitreous humor than the commercial formulation. Thus, while not intending to limit the scope of the invention in any way, the compositions presently disclosed represent a vitreous delivery system which does not require the invasive surgical or injection techniques currently used in the art.

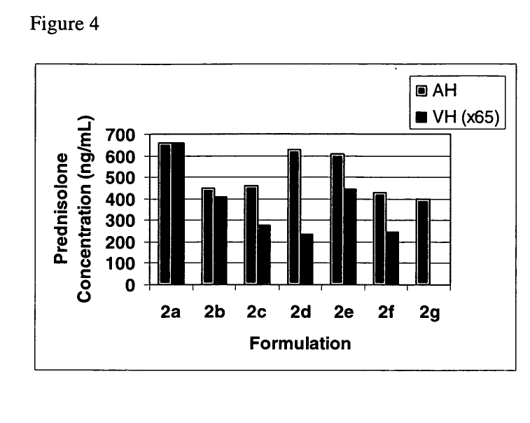

While not intending to limit the scope of the invention in any way, or be bound in any way by theory, this result is particularly unexpected in that the cyclodextrin derivatives appear to have an active role in the transport of the drugs across the aqueous-vitreous barrier. That is, the role of the cyclodextrin derivative appears to be more than simply solubilizing the drug so that a high concentration of the drug will diffuse into the targeted tissue. This hypothesis is clearly supported in the data when one considers that the composition of formula 2g, which contains no cyclodextrin derivative, delivered a measurable concentration of the drugs to the aqueous humor relative to the other formulations, but does not deliver a detectable amount of the drugs to the vitreous humor. By contrast, every cyclodextrin derivative containing formulations delivered a measurable quantity of the drug to the vitreous humor. Thus, the vitreous concentration does not appear to be tied to the aqueous humor concentration, but is related to delivery of the drug by a cyclodextrin derivative. The fact that the concentration of the drugs in the vitreous humor is not determined by the concentration of the drugs in the aqueous humor is also supported by FIG. 4, which compares the concentration of prednisolone in the aqueous humor with that in the vitreous humor for each of the compositions. The vitreous concentration of the drug is multiplied by a factor of 65 for ease of comparison. Clearly, there is no evidence in the data for a correlation between aqueous humor and vitreous humor concentrations of the drug. While not intending to be limited or bound in any way by theory, it follows that the cyclodextrin derivative plays an active role in the delivery of the drug across the barrier. While not intending to be bound in any way by theory, the fact that the commercial formulation contains the same concentration of HPMC as many of the test formulations demonstrates that HPMC is not responsible for the improved delivery seen for the compositions disclosed herein. While not intending to be bound in any way by theory, a person of ordinary skill in the art will recognize that these results suggest that cyclodextrin may be used in delivering many lipophilic drugs to the back of the eye.

### EXAMPLE 3

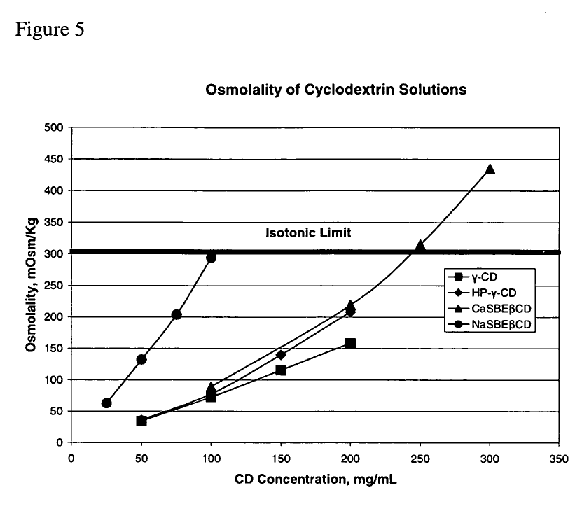

The osmolality of four cyclodextrins was determined as a function of concentration in pure water by the following procedure. Various amounts of cyclodextrins were dissolved in water at ambient room temperature. The results, presented in FIG. 5, demonstrate that sodium salt of sulfobutylether-β-cyclodextrin (NaSBECD) has a significantly higher osmolality than the other βcyclodextrins tested. While not intending to limit the scope of the invention in any way, it appears that the osmolality of NaSBECD in aqueous solution is high enough that its use may be limited at higher concentrations.

### EXAMPLE 4

The aqueous solutions having the composition disclosed in Table 4 were prepared by the following process. Hydroxypropylmethylcellulose (HPMC) was slowly added to water at a temperature of 40° C. with propeller mixing. The heat was removed, and mixing continued while the solution was allowed to cool to room temperature. All of the other excipients except HP-γ-cyclodextrin and prednisolone acetate were added to HPMC solution or pure water, and the mixture was stirred until all solids were completely dissolved. HP-γ-cyclodextrin (HPγCD) was added, and the mixture was stirred until the HPγCD was completely dissolved. Prednisolone acetate was added, and the mixture was stirred for a few minutes. The entire solution was autoclaved at 120° C. for 20 minutes. Stirring continued at room temperature upon removing the solution 10 from the autoclave. The pH was then adjusted by the addition of HCl and/or NaOH, and the solution was filtered through a 0.45 μm cellulose acetate membrane.

TABLE 4
Prednisolone acetate solutions

| | HPγCD, | HPMC, | | | Tonicity | |
|---|---|---|---|---|---|---|
| PA* | % | % | EDTA, % | pH | Agent | Preservative |
| 0.6 | 15 | 0.12 | 0.05 | 4.8 | NaCl | 2 ppm PHMB |
| 0.6 | 25 | 0 | 0.05 | 4.8 | NaCl | 2 ppm PHMB |
| 0.72 | 10 | 0.12 | 0.05 | 4.88 | NaCl | 0.01% CH |
| 0.72 | 10 | 0.12 | 0.05 | 4.8 | NaCl | WSCP |
| 0.73 | 10 | 0.12 | 0.05 | 4.72 | NaCl | 0.01% BAK |
| 0.73 | 10 | 0.12 | 0.05 | 4.76 | NaCl | 5 ppm PHMB |
| 0.8 | 25 | 0 | 0.05 | 4.75 | NaCl | 5 ppm PHMB |
| 0.8 | 25 | 0 | 0.05 | 4.87 | NaCl | 0.01% CH |
| 0.8 | 25 | 0 | 0.05 | 4.78 | NaCl | WSCP |
| 0.81 | 25 | 0 | 0.05 | 4.77 | NaCl | 0.01% BAK |
| 1.2 | 25 | 0.12 | 0.05 | 4.8 | NaCl | 2 ppm PHMB |
| 1.48 | 25 | 0.12 | 0.05 | 4.85 | NaCl | 0.01% CH |
| 1.54 | 25 | 0.12 | 0.05 | 4.72 | NaCl | 0.01% BAK |
| 1.54 | 25 | 0.12 | 0.05 | 4.71 | NaCl | 5 ppm PHMB |
| 1.54 | 25 | 0.12 | 0.05 | 4.71 | NaCl | None |
| 1.55 | 25 | 0.12 | 0.05 | 4.75 | NaCl | 60 ppm WSCP |

CH: Chlorhexidine acetate
PHMB: Polyhexamethylenebiguanidine
WSCP: Water-soluble cationic polymer
BAK: Benzalkonium chloride
Tonicity was adjusted to isotonicity as needed

### EXAMPLE 5

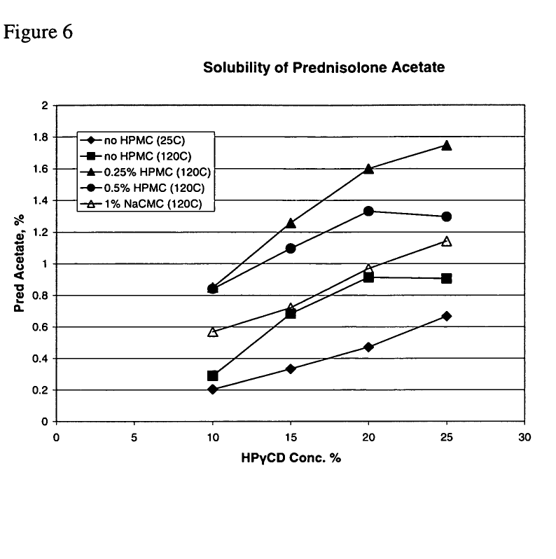

The solubility of prednisolone acetate in hydroxypropyl-γ-cyclodextrin (HPγCD) in the presence of a water-soluble polymer was investigated. The results are presented in FIG. 6. While not intending to limit the scope of the invention in any way, it was surprisingly found that HPγCD is capable of solubilizing over 0.6% prednisolone acetate, which is a therapeutically active concentration. While not intending to limit the scope of the invention in any way, this result demonstrates that in certain circumstances the use of a polymer is not required. However, while not intending to be limiting, these results also show that the use of a polymer can be beneficial under certain circumstances, since both hydroxypropylmethylcellulose (HPMC) and sodium carboxymethylcellulose (NaCMC) enhance the solubility of prednisolone acetate at the polymer concentrations tested. Surprisingly, while not intending to limit the scope of the invention in any way, these results also show that HPMC is superior to NaCMC in improving the solubility of prednisolone acetate, with HPMC having better solubilizing properties at a concentration which is four times lower (FIG. 6).

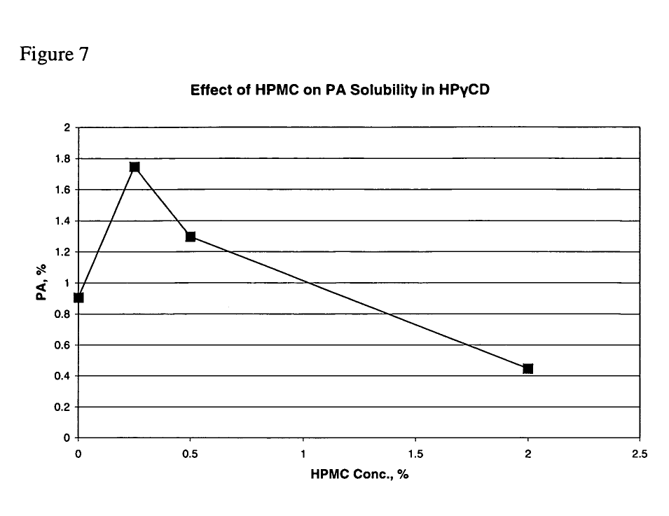

Although the use of the polymer can be beneficial under the proper circumstances, we have surprisingly discovered that there is a range of polymer concentrations which provides the optimum results in terms of prednisolone acetate solubility. FIG. 7 is a plot of the effect of HPMC on the solubility of prednisolone acetate in 25% HPγCD formulations prepared according to the procedure of Example 2. While not intending to limit the scope of the invention in any way, or to be bound in any way by theory, the data in FIG. 7 unexpectedly shows that the maximum solubility of prednisolone acetate occurs where the concentration of HPMC is about 0.25%, and that at higher HPMC concentrations the solubility of prednisolone actually decreases. Thus, while not intending to limit the scope of the invention in any way, for optimal solubility of prednisolone acetate, a formulation should either be prepared without a soluble polymer, or the concentration of the polymer should be less than about 1%.

### EXAMPLE 6

We have unexpectedly found that solutions can be prepared without heating the active ingredient and the cyclodextrin derivative. The solutions having the composition of Table 6, were prepared according to the following procedure.

Part 1

A HPMC solution was prepared by adding the polymer to 40° C. water with propeller mixing. The heat was removed mixing continued while the solution cooled to room temperature.

Part 2

All of the required HP-γ-cyclodextrin was added into 20% of the final volume of water with propeller mixing, and the mixture was stirred to completely dissolve the cyclodextrin. The appropriate amount of prednisolone acetate was added into the solution with propeller mixing, and stirred to completely dissolve the solid. In the solution comprising HPMC, the appropriate amount of the HPMC solution from Part 1 was added. All the other excipients were then added, and the mixture was stirred to completely dissolve all solids. The concentrated solution was then diluted to the final volume, the pH was adjusted with HCl and/or NaCl, and the mixture was filtered through a 0.45 μm cellulose acetate membrane.

TABLE 6
Prednisolone acetate solutions prepared without heating the
cyclodextrin-prednisolone combination

| | | | | | | Tonicity |
|---|---|---|---|---|---|---|
| PA | HPγCD, % | HPMC, % | EDTA, % | Preservative | pH | Agent |
| 1.0 | 25 | 0.12 | 0.05 | PHMB | 4.8 | NaCl |
| 1.0 | 25 | 0 | 0.05 | PHMB | 4.8 | NaCl |

1. A method comprising topically administering a composition to an eye of a mammal, said method being effective in delivering a therapeutically effective amount of a therapeutically active agent to a structure or combination of structures of the eye which include the vitreous humor and structures posterior to the vitreous; said composition comprising:
a. an effective amount of the therapeutically active agent, or a pharmaceutically acceptable salt or prodrug thereof, to provide a therapeutically effective amount of the therapeutically active agent to said structure or combination of structures of the eye, and
b. an effective amount of a cyclodextrin to provide said therapeutically effective amount of said therapeutically active agent to said structure or combination of structures of the eye.

2. The method of claim 1 wherein said mammal is a human.

3. The method of claim 1 wherein said therapeutically active agent, or salt or prodrug thereof, is water-insoluble.

4. The method of claim 1 wherein said therapeutically active agent, or salt or prodrug thereof, is water-soluble.

5. The method of claim 1 wherein said therapeutically active agent is not administered to reduce intraocular pressure.

6. The method of claim 1 wherein said therapeutically active agent is not administered to treat allergic conjunctivitis.

7. The method of claim 1 wherein said therapeutically active agent is not administered to treat dry eye.

8. The method of claim 1 wherein said therapeutically active agent is not administered to treat a condition affecting the front of the eye.

9. The method of claim 1 comprising a β-cyclodextrin derivative.

10. The method of claim 1 comprising a β-cyclodextrin derivative and a water-soluble polymer.

11. The method of claim 1 comprising prednisolone acetate, hydroxypropyl-β-cyclodextrin, and hydroxypropylmethylcellulose.

12. The method of claim 1 comprising a γ-cyclodextrin derivative.

13. The method of claim 5 comprising prednisolone acetate.

14. The method of claim 5 wherein said cyclodextrin derivate is hydroxypropyl-γ-cyclodextrin.

15. The method of claim 5 which further comprises a cellulose derivative.

16. The method of claim 5 which further comprises hydroxypropylmethylcellulose having a concentration less than 1%.

17. The method of claim 5 comprising from 0.05% to 0.4% hydroxypropylmethylcellulose.

18. The method of claim 5 comprising about from 0.1% to 0.25% hydroxypropylmethylcellulose.

19. A pharmaceutical product comprising a solution comprising a therapeutically active agent, or a pharmaceutically active salt or a prodrug thereof, and a cyclodextrin, wherein said solution has an ophthalmically acceptable pH,
a container suitable for dispensing drops of said solution to the eye of a mammal in need of treatment by said prodrug, and
a package which indicates that said product is useful for treatment of a disease or condition affecting the back of the eye.

20. A composition comprising an effective amount of a therapeutically active agent or a pharmaceutically acceptable salt or prodrug thereof, and
an effective amount of a cyclodextrin;
wherein the amount of the therapeutically active agent or salt or prodrug thereof and the amount of the cyclodextrin are effective to deliver a therapeutically effective amount of said therapeutically active agent to a structure or combination of structures of the eye which include the vitreous humor and structures posterior to the vitreous;
wherein the therapeutically effective amount of the therapeutically active agent is delivered by administering said composition topically.

21. The composition of claim 19 wherein said therapeutically active agent is not intended to reduce intraocular pressure.

22. The method of claim 19 wherein said therapeutically active agent is not intended to treat a condition affecting the front of the eye.

23. The composition of claim 20 comprising from 0.1% to 2% prednisolone acetate and from 1% to 30% of the cyclodextrin.

24. The composition of claim 23 comprising a β-cyclodextrin derivative.

25. The composition of claim 23 comprising a γ-cyclodextrin derivative.
